(19) United States
(12) Patent Application Publication
(10) Pub. No.: US 2018/0326061 A1
(43) Pub. Date: Nov. 15, 2018

(54) MODIFIED TRANSDERMAL DELIVERY DEVICE OR PATCH AND METHOD OF DELIVERING INSULIN FROM SAID MODIFIED TRANSDERMAL DELIVERY DEVICE

(71) Applicant: BKR IP Holdco LLC, Broomall, PA (US)

(72) Inventor: Bruce K. REDDING, JR., Broomalll, PA (US)

(21) Appl. No.: 15/774,225

(22) PCT Filed: Nov. 7, 2016
(86) PCT No.: PCT/US16/60854

§ 371 Date: May 7, 2018

Related U.S. Application Data
(60) Provisional application No. 62/285,668, filed on Nov. 6, 2015.

(51) Int. Cl. A61K 41/00; A61K 9/00; A61K 9/70; A61K 38/28; A61M 37/00

(52) U.S. Cl. CPC A61K 41/0047; A61K 9/0009; A61K 9/7084; A61M 2205/50; A61M 37/0092; A61M 2037/0007; A61K 38/28

(57) ABSTRACT

The invention is a means to provide enhanced delivery of a drug from a transdermal patch, employing a screen filter at the bottom of the patch which contacts the skin. The screen filter enables the drug from the patch to become deposited onto the surface of the skin in a series of droplets, spaced in such a manner as to match the skin's pores. The drop deposition of the patch increases the speed of delivery of the patch, whether an active or a passive patch form.

### PRIORITY CLAIM, CROSS REFERENCE TO RELATED APPLICATIONS AND INCORPORATION BY REFERENCE

This application is related to, claims priority under, and claims the benefit of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790: “METHOD AND APPARATUS FOR MEASURING THE DOSE REMAINING UPON A TRANSDERMAL DRUG DELIVERY DEVICE”. Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US/39236.

This application hereby incorporates herein by reference the subject matter disclosed in the abstract, written descriptions, the drawings and claims, in their entireties of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US/39236.

### FIELD OF THE INVENTION

The present invention relates generally to transdermal patches and bandages, also known as drug delivery devices. The present invention relates more specifically to a modified transdermal patches and bandages which incorporate a separator, which could be a screen filter at the section which comes into contact with the skin, wherein the separator, which could be a screen filter acts to form droplets of the drug as the drug is released from the patch, for the purposes of speeding the drug delivery and increasing the longevity of the patch.

### BACKGROUND OF THE INVENTION

In the prior art, transdermal drug delivery systems employ a medicated device or patch which is affixed to the exposed surface of the skin of a patient. The patch allows a medicinal compound contained within the patch to be absorbed into the skin layers and finally into the patient's blood stream. Transdermal drug delivery avoids the need and the pain associated with drug injections and intravenous drug administration. Transdermal drug delivery also avoids gastrointestinal metabolism of administered drugs, reduces the elimination of drugs by the liver, and provides a sustained release of the administrated drug. Transdermal drug delivery also enhances patient compliance with a drug regimen because of the relative ease of administration and the sustained release of the drug.

Several medicinal compounds are not suitable for transdermal drug delivery since they are absorbed with difficulty through the skin due to the molecular size of the drug or to other bioadhesion properties of the drug. In these cases, when transdermal drug delivery is attempted, the drug may be found pooling merely on the outer surface of the skin and not permeating directly through into the blood stream. Once such example is insulin, which in the prior art has been found difficult to administer by means of transdermal drug delivery.

Some of the most critically needed medications are presently administered either by injection or oral dosage forms. In particular, chemotherapeutic agents are administered in increased dosages because of their need to survive degradation in the gastrointestinal tract. Many critical treatments for AIDS require a cocktail of drugs taken orally in solid dosage forms, several times a day to be effective. These medications are not suitable for transdermal drug delivery use because of the extensive dosing requirement, the inability of the drug molecule to remain stable in a transdermal form. Moreover, the unsuitability for transdermal to skin transfer of the drug leading to low bioabsorbance of the drug across the skin layers.

Generally, conventional transdermal drug delivery methods have been found suitable only for low molecular weight medications such as nitroglycerin for alleviating angina, nicotine for smoking cessation regimens, and estradiol for estrogen replacement in post-menopausal women. Larger molecular medications such as insulin (a polypeptide for the treatment of diabetes), erythropoietin (used to treat severe anemia) and gamma-interferon (used to boost the immune systems cancer fighting ability) are all compounds not normally effective when used with transdermal drug delivery methods of the prior art.

There are three basis designs to transdermal patch products:

### 1. Reservoir Type Patch:

Characterized by the inclusion of a liquid reservoir compartment containing a drug solution or suspension, which is separated from a release liner by a semi permeable membrane and an adhesive.

Commercial examples include: Duralgesic® (Fentanyl), Estraderm® (estradiol) and Transderm-Nitro® (Nitroglycerin).

### 2. Matrix Type Patch:

Similar to the Reservoir Type Patch design but has two distinguishing characteristics:
1. The drug reservoir is provided within a semisolid formulation.
2. There is no membrane layer.
Commercial Examples include Habitol® (Nicotine), Nitrodisc® (Nitroglycerine and ProStep® (Nicotine)

### 3. Drug-in-Adhesive Type Patch: DIA

Characterized by the inclusion of the drug directly within the skin-contacting adhesive (Wick 1988). In this design the adhesive fulfills the adhesion-to-skin function and serves as the formulation foundation, containing the drug and all the excipients. (Wilking 1994). This category also has two sub-sections: Monolithic and Multilaminate.

Commercial examples include Monolithic DIA: Climara® (Estradiol), Multilaminate DIA: Nicoderm® (Nicotine)

The DIA patch design has several advantages in reducing the size of the overall patch and provides a more concentric seal upon the skin. DA patches tend to be more comfortable to wear and very thin. A typical DIA patch is 165 to 200 Um thick. Major disadvantages include a longer drug delivery profile. The release of the drug from a DIA patch follows first order kinetics, that is, it is proportional to the concentration of drug within the adhesive. As the drug is delivered from the DIA patch the drug concentration will eventually begin to fall. The delivery rate therefore falls off over time and this fact needs to be considered in the clinical evaluation phase of development.

A major problem with all major forms of transdermal patches is the intermingling of the drug with adhesive compositions. These result in new profiles and in many instances the drug is degraded through the interaction with the adhesive composition. The chemistry of the adhesive can alter the stability, performance and function of certain drugs.

Additionally there are limits to the molecule size of drugs, which can be delivered via a passive system. Typically drug candidates are below 500 Daltons for DIA patches and below 1,000 Daltons for Matrix or Reservoir patches, even through the use of skin enhancers.

### Electronically Assisted Transdermal Devices

There are several approaches used to electronically assist in transdermal delivery including iontophoresis and ultrasound. These systems are designed to either increase the flow of metallic based drugs across the stratum corneum or to microporate the skin or allow the delivery of macromolecules across the stratum corneum into the dermis or underlying tissue. Such electronically assisted transdermal drug delivery devices (TDD”) often use an outside electronic system, which is not connected to a drug-containing patch or the patch has electrodes within it to assist in ionic transfer. Direct connection to a disposable transdermal patch is often impractical because the electrodes or the ultrasonic transducer system is not disposable.

To solve the problem of electronically assisted transdermal drug delivery systems, and enabling such systems to become more portable or wearable by the patient, and in consideration of conventional patch designs wherein drug contamination or denaturing may be caused through interaction with an adhesive or polymer component within the patch design a new transdermal patch, the subject of this invention, was developed.

The use of adhesives, which directly contact the drug, is eliminated in this design. Adhesives may be used in the border of the patch but the DIA, Matrix or Reservoir designs are discarded in favor of an absorbent pad which is held in place in the patch of this invention which also employs a rate control semi-permeable film to provide both on-off functions to the patch and dosing control. The Patch of this invention is also fitted with a snap to enable the patch to connect easily to an ultrasonic emitter. This design enables the more expensive ultrasonic emitter to be retained for future use while the Modified Transdermal Patch is disposable.

Patches are designed to provide either passive or active delivery platforms.

The skin has evolved as a formidable barrier against invasion by external microorganisms and against the prevention of water loss. Notwithstanding this, transdermal drug delivery systems have been designed with the aim of providing continuous controlled delivery of drugs via this barrier to the systemic circulation. There are numerous systems now available that effectively deliver drugs across the skin. These include reservoir devices, matrix diffusion-controlled devices, multiple polymer devices, and multilayer matrix systems. This review article focuses on the design characteristics and composition of the main categories of passive transdermal delivery device available.

Mechanisms controlling release of the active drug from these systems as well as patch size and irritation problems will be considered. Recent developments in the field are highlighted including advances in patch design as well as the increasing number of drug molecules now amenable to delivery via this route. From the early complex patch designs, devices have now evolved towards simpler, matrix formulations. One of the newer technologies to emerge is the delivery-optimized thermodynamic (DOT) patch system, which allows greater drug loading to be achieved in a much smaller patch size. With the DOT technology, drug is loaded in an acrylic-based adhesive. The drug/acrylic blend is dispersed through silicone adhesive, creating a semi-solid suspension. This overcomes the problem with conventional drug-in-adhesive matrix patches, in which a large drug load in the adhesive reservoir can compromise the adhesive properties or necessitate a large patch size.

Transdermal drug delivery remains an attractive and evolving field offering many benefits over alternative routes of drug delivery. Future developments in the field should address problems relating to irritancy and sensitization, which currently exclude a number of therapeutic entities from delivery via this route. It is likely that further innovations in matrix composition and formulation will further expand the number of candidate drugs available for transdermal delivery.

### Active Transdermal Drug Delivery Market Dynamics

The administration of therapeutic drugs via e advantages of transdermal drug delivery for improving patient compliance, particularly for the treatment of chronic conditions, are well known. But growth of transdermal delivery has been restricted by the need to limit candidate drugs to molecules small enough to effectively pass through the stratum corneum, a limitation that excludes passive transdermal patches as a viable option for the growing number of protein and peptide therapeutic compounds that will represent an increasing share of future NCEs. New technologies that employ energy or mechanical designs to affect drug transport through the skin are expanding the type and number of drug candidates that are viable for transdermal delivery. Evolving active transdermal systems will be well-positioned to address a significant segment of the large-molecule biological drugs expected to emerge from the convergence of automated discovery and genome mapping. As designs shrink in size and become more patient-friendly, opportunities for active transdermal delivery will increase.

Active Transdermal Technology Overview

Market Drivers for Transdermal Delivery

Excipients and Penetration Enhancers

Competitive Landscape

Factors Limiting Growth

Active Transdermal Technologies

Electrical Current

Iontophoresis

Electroporation

Microporation

Lasers

Mechanical Arrays

Radio Frequency

Thermal/Heat

Ultrasound

Active Transdermal Design Factors

Drug Formulation Factors

Proprietary Delivery vs. 3rd Party Patches

Dosing and Rate Factors

Biocompatibility

### Delivery Pattern Upon the Skin

Typically upon release from a standard TDD the drug will form a large spot upon the skin. This spot formation effect slows the absorption rate through the skin and can waste the drug as a large quantity simply does not permeate through the skin. See FIG. 16.

### SUMMARY OF THE PRESENT INVENTION

### Drug Pooling Slows Drug Absorption from Patch

To improve the speed of drug absorption upon liberation from the patch, the Delivery Pattern of the insulin is directed to enter the skin at the site of the skin pores. A separator, which could be a filter at the bottom of the Trans-Insulin patch reduces the drug to miniature droplets which approximate the spacing for the skin's pore structure. As a result the insulin is absorbed more completely into the skin and at a faster pace. See FIG. 5B which is a drawing of a Photograph where the insulin is marked with a blue dye, and is more readily absorbed at the pore distribution sites.

The insulin droplet approach reduced the quantity of insulin needed to be stored within the TDD and increased the speed of absorption into the skin. In the original design of the Patch-Cap it took 5 hours of constant ultrasound to reduce the glucose by just 40 points. In the new design, using the Dot Pattern, the glucose was dropped 40 points in 30 minutes for 87% of the volunteers tested.

An objective of the modified transdermal patch is to optimize, speed and improve the efficiency of transdermal drug transport of both small and large molecule drugs, particularly insulin, through the skin in either a passive or through an active patch design such as in conjunction with the U-Strip device and any other method of employing ultrasound in transdermal drug delivery. Ultimately, this innovative development could extend the types and number of drugs given transdermally as well as allow combinations of drugs to be given safely and accurately, by simply reducing the amount of drug which gets deposited and stays upon the surface of the skin.

Accordingly, a purpose of this invention is to provide a modified transdermal patch for enhancing transdermal drug delivery by the use of at least one separator, which could be a filter or mesh screen which has been placed at the bottom of the patch where it connects to the skin. The mesh screen separates the drug into droplets which fall onto the skin in a pattern approximating the skin pore pattern upon the skin. The droplet pattern is absorbed at a faster rate through the skin with less waste of the drug which in a normal patch is simply deposited into a pool upon the skin.

This is especially functional with the transdermal delivery of insulin from a patch which is subjected to ultrasonic excitation.

The present invention is a transdermal delivery device, bandage or patch designed with at least one separator, which could be at least one screen or filter at the bottom of a patch for the formation of droplets of the drug exiting the patch.

An object of the invention is a delivery device, which could be a transdermal patch which has at least one separator, which could be at least one mesh screen under the device, for reducing the substance, which could be a drug, delivered form the device, which could be a patch, into droplet form, wherein the droplet release is matched according to the pattern of openings in living tissue, which could be pores, and can thereby enable faster penetration through the tissue, which could be skin, of the substance, which could be one or more drugs, with less waste and greater efficiency.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 is a diagram illustrating the design of a Reservoir Transdermal Patch

FIG. 2 is a Drug-In-Adhesive Matrix Construction Patch.

FIG. 3 is an illustration of the structure of human skin.

FIG. 4A is an illustration which shows that conventional transdermal patches tend to release their drug content, either through passive delivery or through active delivery means, into a pool which first rests upon the surface of the skin and which is then slowly drawn into the skin over time.

FIG. 4B is a drawing of a photograph of insulin deposited upon the surface of the skin from an active transdermal patch, wherein the insulin, marked in this instance with a red dye marker, formed a pool which rested upon the surface of the skin and was not readily absorbed into the skin.

FIG. 5A is a schematic illustration of the invention, which is separator of a layer of mesh screen or a filter added to the bottom of a transdermal patch, for the purpose of producing droplets of the drug onto the skin's surface, wherein those droplets can be more readily absorbed into the skin at a faster rate and with less drug waste.

FIG. 5B illustrates the drug deposition pattern of the drug when the patch uses a separator of a screen filter or mesh pattern to deposit the drug to the pores of the skin, matching the pattern of the pore disposition.

FIG. 5C is a schematic illustration which is separator at the proximal end of a delivery device the which produces droplets of the substance onto the live tissue surface.

FIG. 6 is a Top View depiction of a flexible transdermal patch design modified to use the mesh screen at the bottom of the batch. This particular design uses an absorbent pad to hold the drug and the drug is liberated under an Active control fashion using ultrasound.

FIG. 7 is the Bottom View of a flexible transdermal patch design modified to use the mesh screen at the bottom of the patch.

FIG. 8A is an Active transdermal delivery device termed a Patch-Cap, designed to mate with a transducer coupler for the purpose of delivering insulin, employing a Screen Mesh fabric at the bottom of the transdermal Patch-Cap.

FIG. 8B illustrates how the Transducer coupler is mated to the Patch-Cap illustrated in FIG. 8A.

FIG. 9 is an illustration of a screen mesh fabric.

FIG. 10 is a Screen Mesh Filter Cap which could be attached to a flexible transdermal patch or Patch-Cap transdermal delivery device.

FIG. 11 is a schematic of the Screen Mesh Filter Cap.

FIG. 32-F is the connection to a volunteer for Experiment-1, a test of transdermal delivery device, a Patch-cap, loaded with insulin and powered by ultrasound, with and without the use of a mesh screen.

FIG. 32-F.2 is an illustration of Experiment-1, a test of a transdermal delivery device, a Patch-Cap, loaded with insulin and powered by ultrasound, with the use of a mesh screen, as it was affixed to a test subject during the experiment.

FIG. 32-F.3 is an illustration of the equipment used in Experiment-1.

FIG. 32-F.4 is an illustration of a patch-Cap loaded with 100 units of Lispro insulin, used in Experiment-1, a test of a transdermal delivery device, a Patch-cap, loaded with insulin and powered by ultrasound, with the use of a mesh screen.

FIG. 32-G illustrates a Portable transdermal delivery device, a Patch-cap, powered by ultrasound for the delivery of insulin in the treatment of diabetes.

### DESCRIPTION OF THE INVENTION

It is to be understood that the figures and descriptions of the present invention have been simplified to illustrate elements that are relevant for a clear understanding of the present invention, while eliminating, for purposes of clarity, many other elements found in conventional ultrasonic substance delivery systems. Those of ordinary skill in the art will recognize that other elements are desirable and/or required in order to implement the present invention. However, because such elements are well known in the art, and because they do not facilitate a better understanding of the present invention, a discussion of such elements is not provided herein.

As used herein, the term “substance” may include, but are not limited to, any substance, solution or suspension including, but not limited to, a medicinal or non-medicinal substance which may be transported through a live surface or live membrane, including, but not limited to, live tissue and other types of live membranes. The term “delivery device” includes transdermal patches and bandages. The term “proximal” means toward the end of a delivery device where the substance is released from the device. The term “distal” means toward the end of the device that is away from where the substance is released from the device.

Structure of Human Skin and Drug Transport Dynamics.

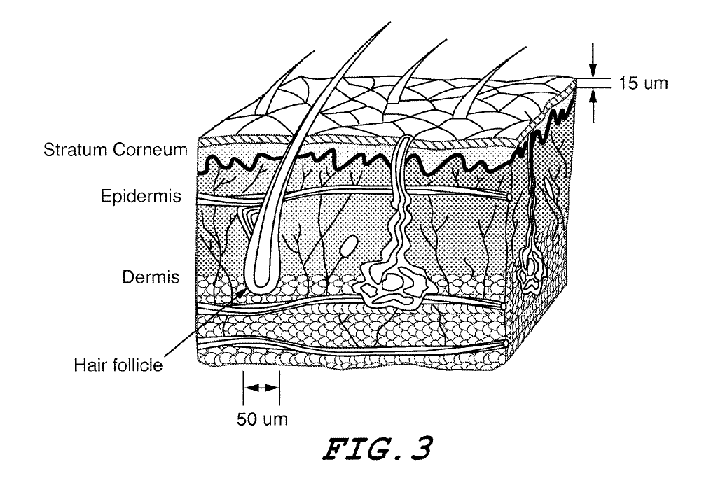

FIG. 3 illustrates the structure of human skin. Essentially there are three pathways through the skin into the bloodstream:
1. Breaching the Stratum Corneum.
2. Passing pharmaceutical agent through pores in the skin.
3. Passing a pharmaceutical agent through the skin by following the hair follicle to the hair root, and from there into the vascular network located at the base of the hair root.

FIG. 1 is a diagram illustrating the design of a Reservoir Transdermal Patch. Characterized by the inclusion of a liquid reservoir compartment containing a drug solution or suspension, which is separated from a release liner by a semi permeable membrane and an adhesive.

Commercial examples include: Duralgesic® (Fentanyl), Estraderm® (estradiol) and Transderm-Nitro® (Nitroglycerin).

A Matrix Type Patch is Similar to the Reservoir Type Patch design but has two distinguishing characteristics:
1. The drug reservoir is provided within a semisolid formulation.
2. There is no membrane layer.
   Commercial Examples include Habitol® (Nicotine), Nitrodisc® (Nitroglycerine and ProStep® (Nicotine)

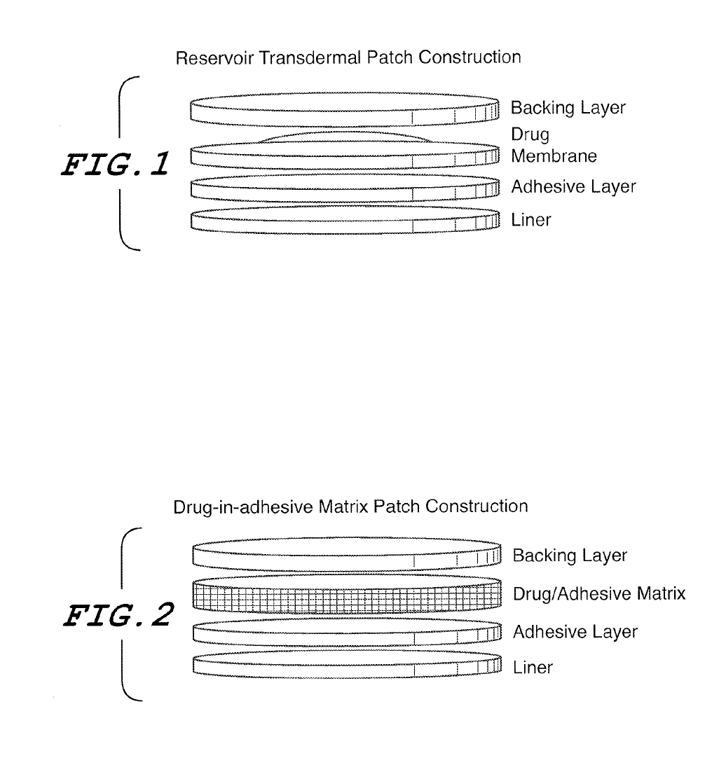

FIG. 2 is a Drug-In-Adhesive Matrix Construction Patch also known as a DIA Patch. Characterized by the inclusion of the drug directly within the skin-contacting adhesive (Wick 1988). In this design the adhesive fulfills the adhesion-to-skin function and serves as the formulation foundation, containing the drug and all the excipients. (Wilking 1994). This category also has two sub-sections: Monolithic and Multilaminate.

Commercial examples include Monolithic DIA: Climara® (Estradiol), Multilaminate DIA: Nicoderm® (Nicotine)

The DIA patch design has several advantages in reducing the size of the overall patch and provides a more concentric seal upon the skin. DIA patches tend to be more comfortable to wear and very thin. A typical DIA patch is 165 to 200 Um thick. Major disadvantages include a longer drug delivery profile. The release of the drug from a DIA patch follows first order kinetics, that is, it is proportional to the concentration of drug within the adhesive. As the drug is delivered from the DIA patch the drug concentration will eventually begin to fall. The delivery rate therefore falls off over time and this fact needs to be considered in the clinical evaluation phase of development.

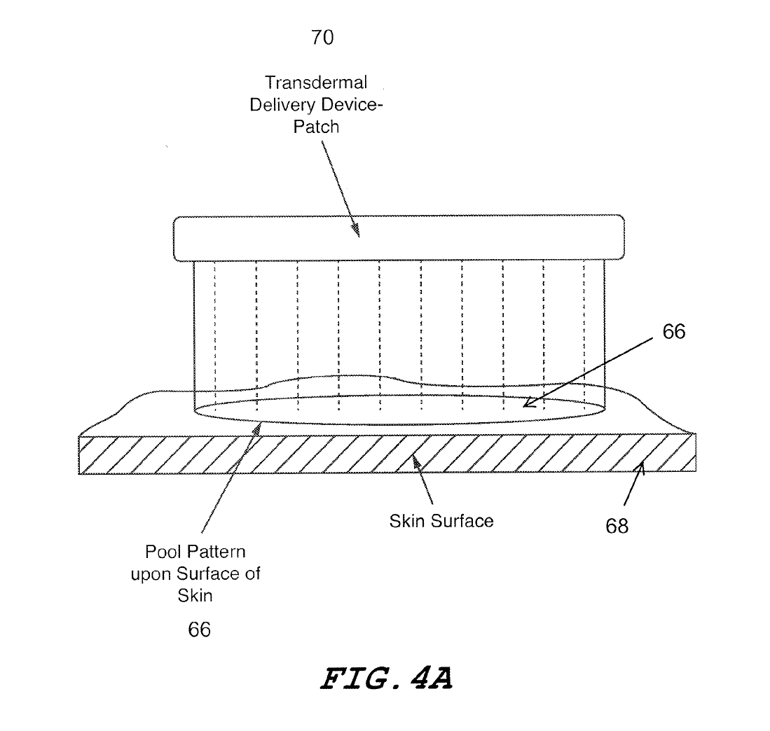

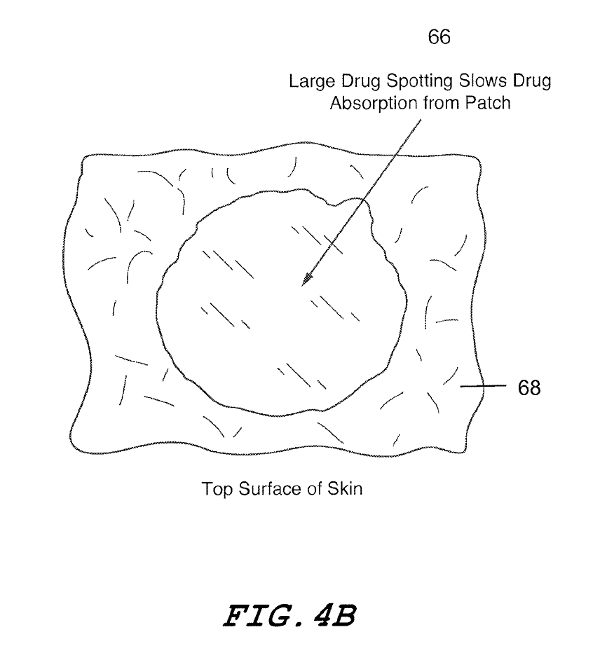

A major problem with all major forms of transdermal patches 70 is the deposition of the drug in a pool 66 upon the skins surface upon existing the patch as seen in FIG. 4. The typical release pattern is uncoordinated from the patch and the drug tends to pool upon the surface of the living tissue 68, which could be skin. FIG. 4B is a photograph of the drug, in this instance mixed with a red dye, which pools 66 upon the skin surface after release from the transdermal delivery device or patch. In this example insulin (Humalog Reg, U-100 supplied by Eli Lilly Company) has been mixed with a red dye. Insulin if nearly 6,000 Da in molecular=weight and is not readily absorbed into the skin through passive means. It requires an Active delivery mechanism such as ultrasound, heat therapy to the skin, laser, or iontophoresis. These Active delivery system tend to force large molecule drugs, such as insulin through the skin pores, either through the pores surrounding a hair follicle or through the sweat pores, as illustrated in FIG. 3.

These result of the pooling effect upon drug release can be very dramatic:
a) The Pooling wastes a good portion of the drug. A large quantity of drug can remain upon the skin surface and is not readily absorbed into the skin, either passively or actively.
b) The pooled drug remains will suffer varying release profiles as it departs the patch. The pool begins to slow the release of more of the drug from the patch as the initially pooled drug tends to form a resistive barrier to additional liberation. As a result the delivery profile of the patch slackens off in later hours.
c) Pooled drug upon release increases the chance of contamination with impurities in the air, from under the patch or from germs or existing chemicals already on the skin's surface (the result often of the use of improperly cleansed cosmetic lotions and moisturizers upon the skin). The longer the pool exists the greater the chance for contamination.

To combat these problems, a separator 72 is placed on the delivery device 76 such that the substance 74 is separated into droplets 78 when deposited on the living tissue 68, which could be skin. It is to be understood that providing any separator known in the art or to be discovered is included in the invention as a method of providing a substance to a living tissue, which could be the skin. The invention further includes separating a substance into droplets before it engages with the living tissue, which could be skin, so that it engages the skin in the form of separate droplets.

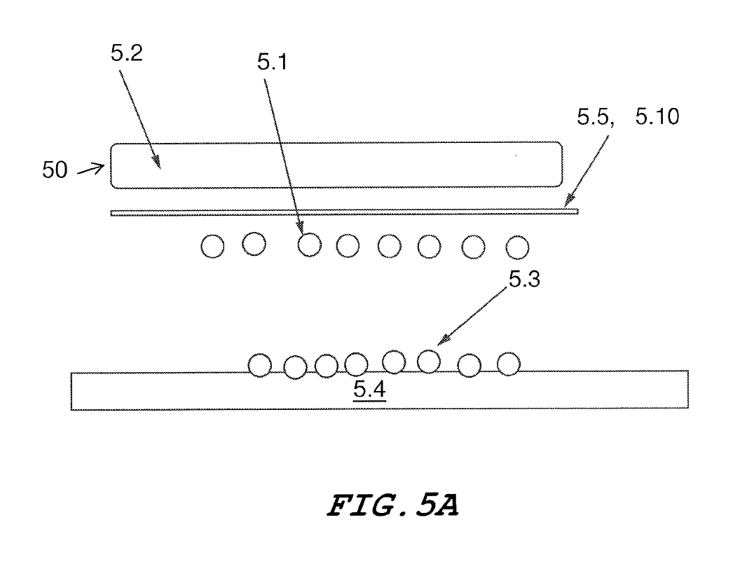

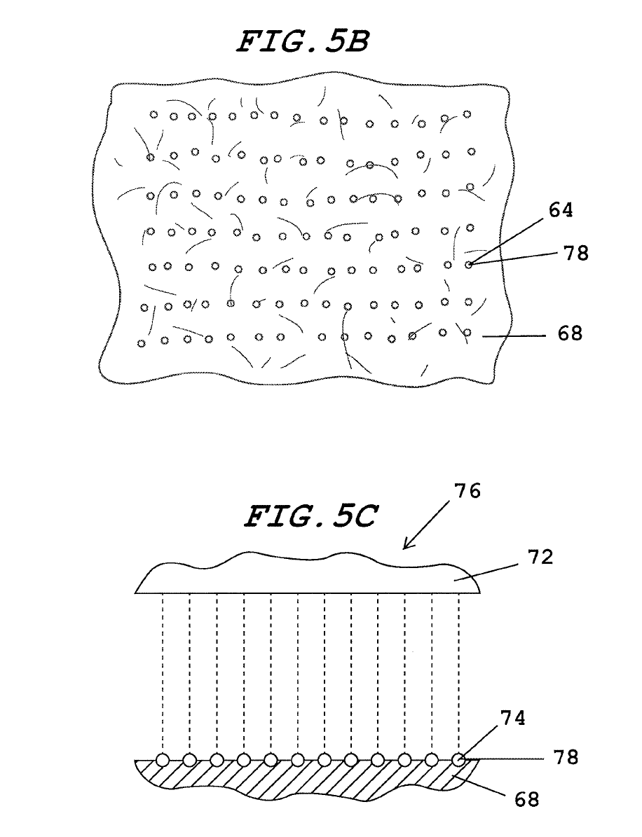

In some embodiments, the separator can be a mesh screen. As shown in FIG. 5A, the use of a simple screen mesh (5.5) is placed at the bottom, or proximal end 50 of the transdermal patch (5.2) and can reduce or eliminate drug pooling completely. The drug (5.1) upon exiting the patch (5.10) forms into droplets (5.3) as the drug passes through the mesh and is then deposited upon the surface of the skin (5.4). No drug pool forms.

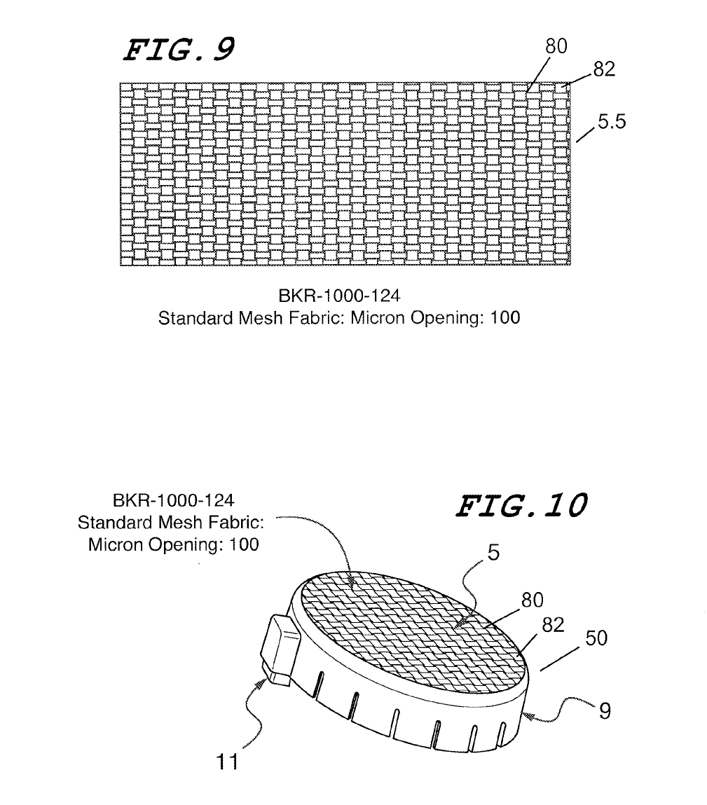

A screen fabric is illustrated in FIG. 9. There is a spacing 80 between the threads 82 or fibers of the mesh which allows the substance to penetrate through the spacing upon release from the patch.

Common materials used in the mesh include:
a) Polyester,
b) Polypropylene,
c) Nylon

### Weave Patterns can be:

a) 100×100 fibers per inch vertical & horizontal.
b) 72×72 warp and weave
c) 72 fibers per inch vertical & horizontal.
d) Or other possible mesh configurations

The size of the opening within the mesh determines the dimensions and weight of the substance or drug droplet upon release. FIG. 9 is an illustration (not to scale) of a screen mesh fabric using 100×100 fibers per inch vertical & horizontal. This spacing of the mesh tends to produce droplet sizes upon the skin, of a substance or drug passing through the mesh screen, approximately 50 microns in diameter. The skin pores of the body are generally 50 microns in diameter, so the substance or drug is deposited to match the pore opening size, where it can avoid pooling upon the skin and as a match to the pore's diameter, the substance or drug is more speedily and effectively absorbed at the pore site.

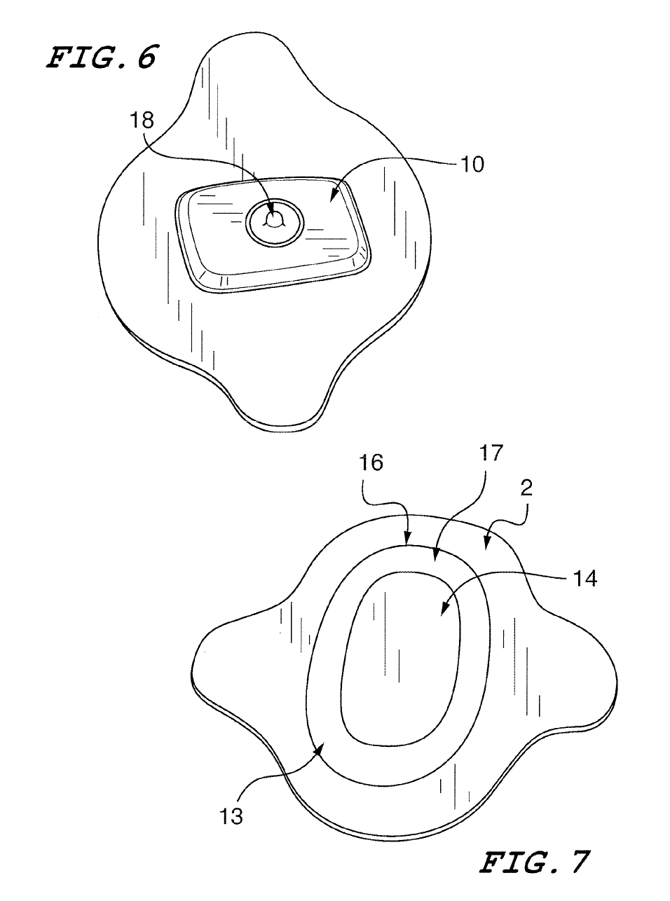

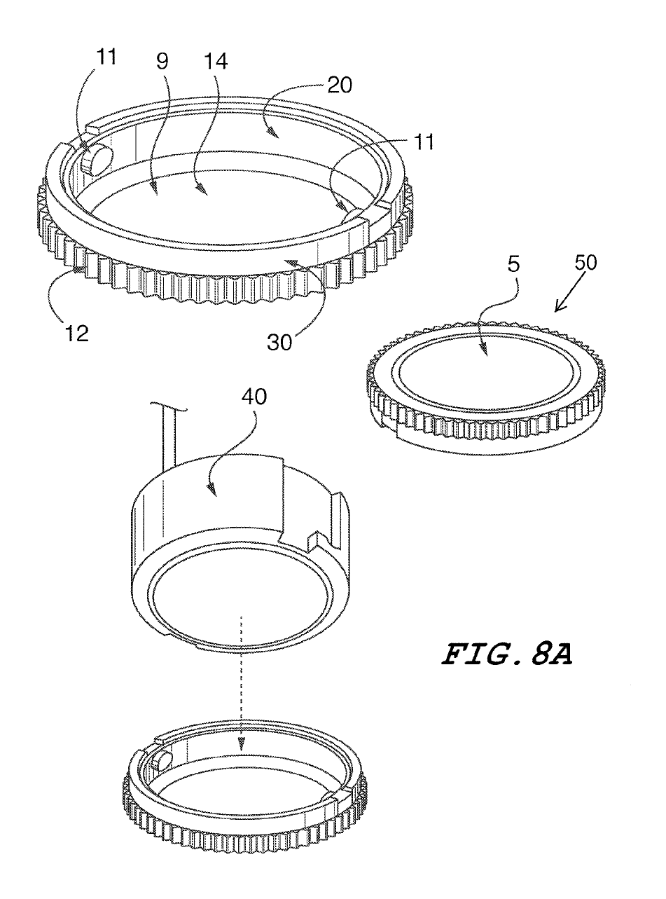

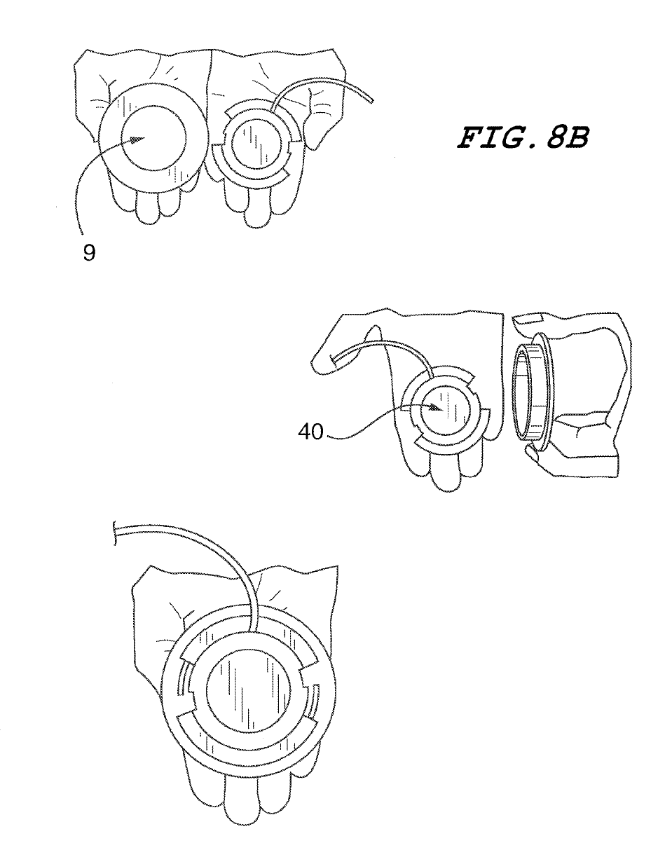

FIG. 5B is a drawing of a photograph of insulin marked with a blue dye 64 that has exited a Patch-Cap, a particular form of transdermal delivery device, an Active transdermal system, designed especially for transdermal insulin delivery. The Patch-Cap is illustrated in FIGS. 8A and 8B. In additional a flexible transdermal patch, designed with one or more absorbent pads or layers of absorbent material to absorb the drug and hold it until liberated by ultrasound is depicted in FIGS. 6 and 7.

In FIG. 5B it can be seen that the substance which could be a drug upon passing through the mesh is developed into small droplets 78, highlighted on the photograph with a blue dye 64. The type of mesh chosen enabled the drug droplets to form directly over the pores of the skin, where the absorption rate is enhanced significantly. There is virtually no pooling of the substance which could be a drug which indicates that more of the substance is actually absorbed into the living tissue 68 which could be skin and there is less waste.

FIG. 6 is a Top View depiction of a flexible transdermal patch design modified to use the mesh screen at the bottom of the patch. This particular design uses one or more layers of absorbent material which could be one or more absorbent pads to hold the substance, which could be a drug and the substance is liberated under an Active control fashion using ultrasound. In this design the flexible patch may be used passively with low molecular weight drugs, generally below 1,000 Daltons. On the top of the patch 10 is a snap 18 which can attach the patch to an ultrasonic transducer device, which in turn sends an ultrasonic transmission through the patch and liberates the drug onto the surface of the skin. The backbone 2 or border of the patch which comes into contact with the skin has an adhesive layer to stick the patch to the skin's surface. The adhesive does not come into contact with the drug directly. The one or more layers of absorbent material which could be one of more absorbent pads 14 is placed with a bordered well 17 which isolates it from contact with the adhesive layer 2. The mesh screen 16 is placed across the drug reservoir 13 and over the absorbent material. Using this construction a transdermal patch forms minute droplets of the drug upon the skin's surface as depicted in FIG. 5B, either through passive or active means. This design patch is especially suited for ultrasonic drug delivery.

FIG. 8A is an Active transdermal delivery device termed a Patch-Cap, designed to mate with a transducer coupler for the purpose of delivering insulin, employing a Screen Mesh fabric at the bottom of the transdermal Patch-Cap.

FIG. 8B illustrates how the Transducer coupler is mated to the Patch-Cap illustrated in FIG. 8A.

FIG. 8A is an illustration of a Transdermal Patch Cap designed to deliver insulin transdermally using ultrasonic propagation. The Patch Cap includes one or more layers of an absorbent material which could be one or more absorbent pads 14, which is placed within a holder, the outer snap ring 30. It is locked into place by an inner snap ring 20 and then is used to absorb a drug, particularly insulin. The Cap has threaded sides usually 12 and a cap connector grove 11 which fits into an ultrasonic emission transducer coupler 40. The mesh screen 5 is placed across the absorbent material 14 at the bottom, or proximal end 50, of the patch cap. Using this construction a transdermal patch-cap forms minute droplets of the drug upon the skin's surface as depicted in FIG. 5B, either through passive or active means. This design patch is especially suited for ultrasonic drug delivery.

FIG. 10 illustrates an alternative Screen Mesh Filter Cap 9, which can be applied to an Active Transdermal delivery System such as the Patch-Cap used for ultrasonic transdermal delivery as depicted in FIG. 10. A mesh screen 5 is placed across the top of the cap 9. The Filter Cap 9 can be fitted onto a patch or a delivery device through a connector 11. The Screen Mesh Filter Cap 9 is designed to be an attachment to any active transdermal delivery system.

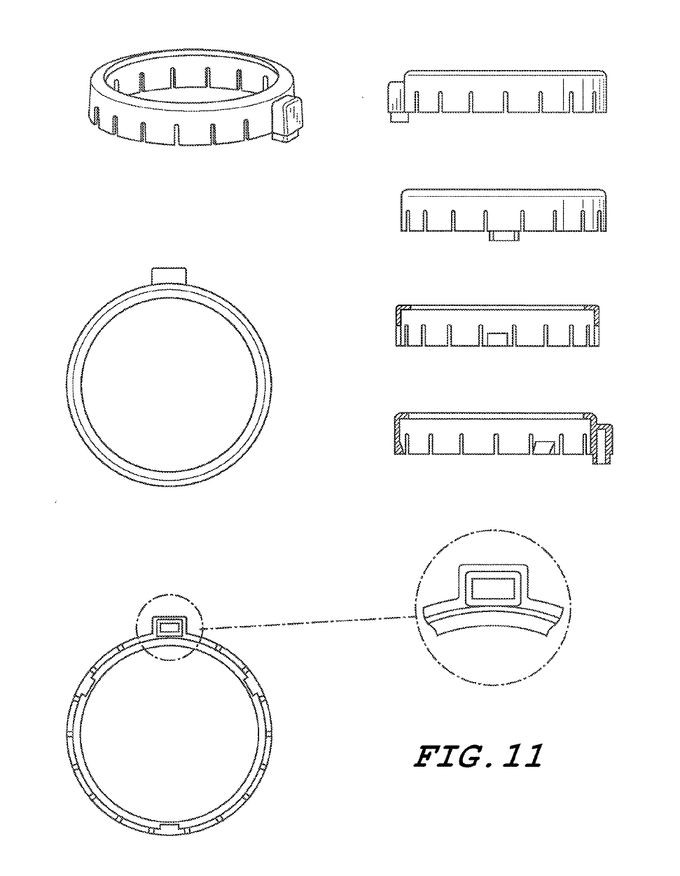

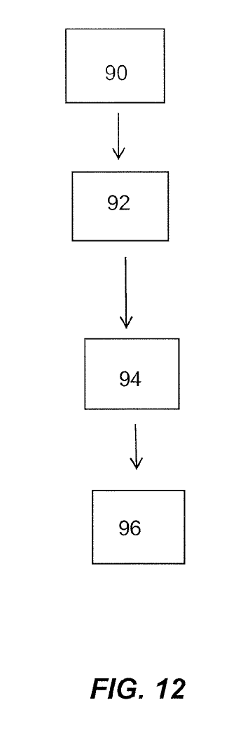

FIG. 11 is an engineering drawing showing the dimensions and construction of the Screen Mesh Filter Cap 9.

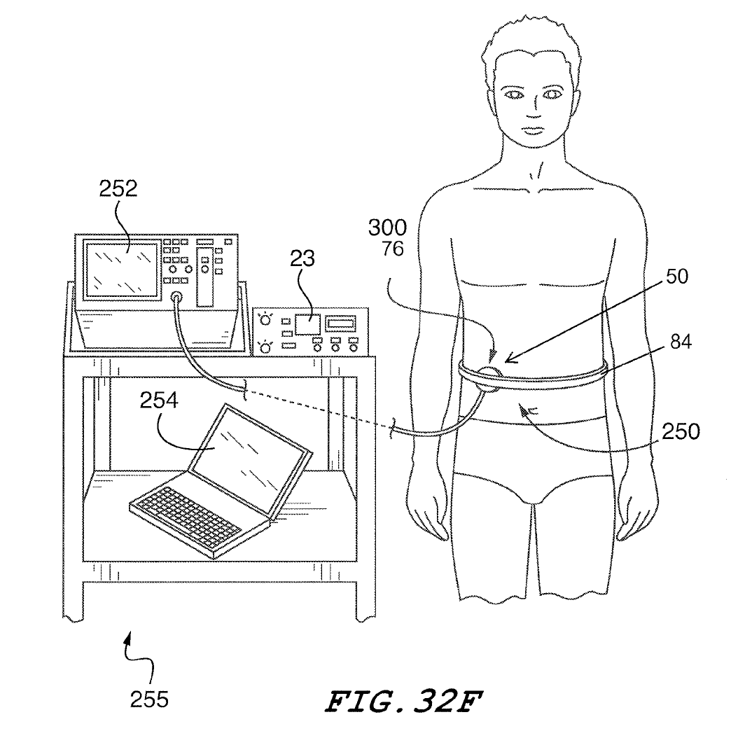

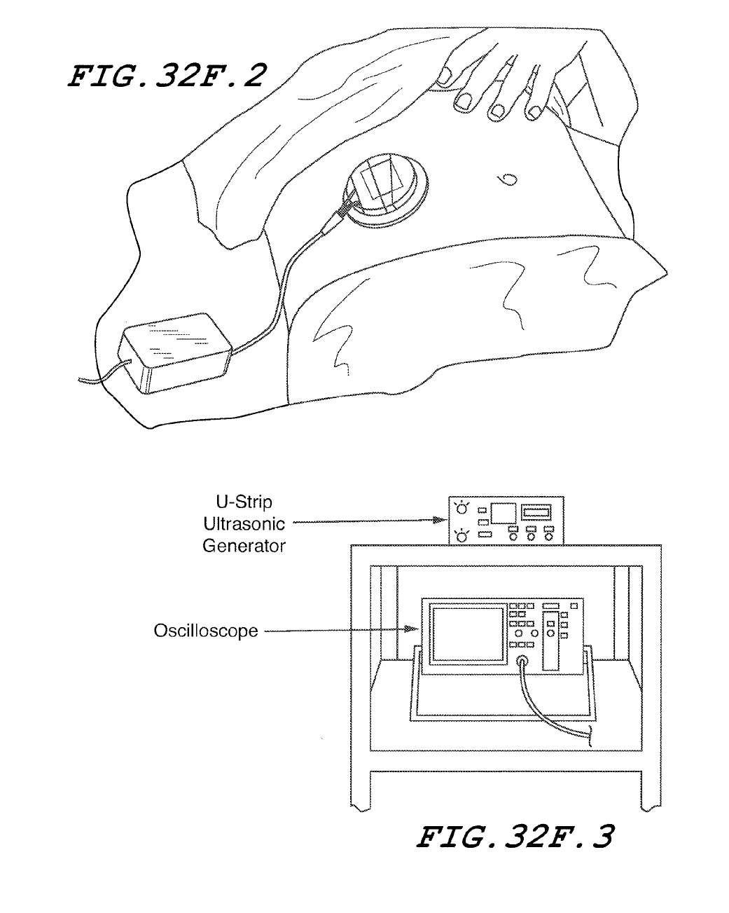

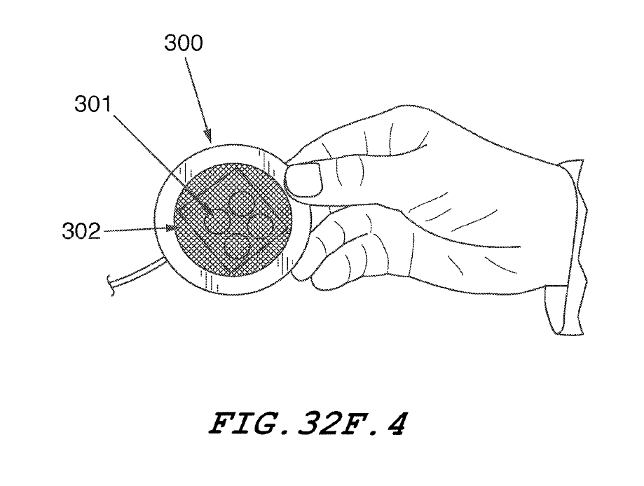

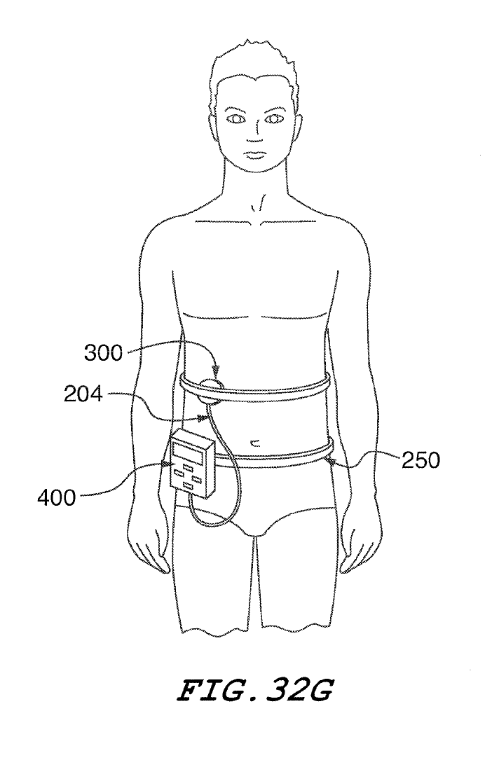

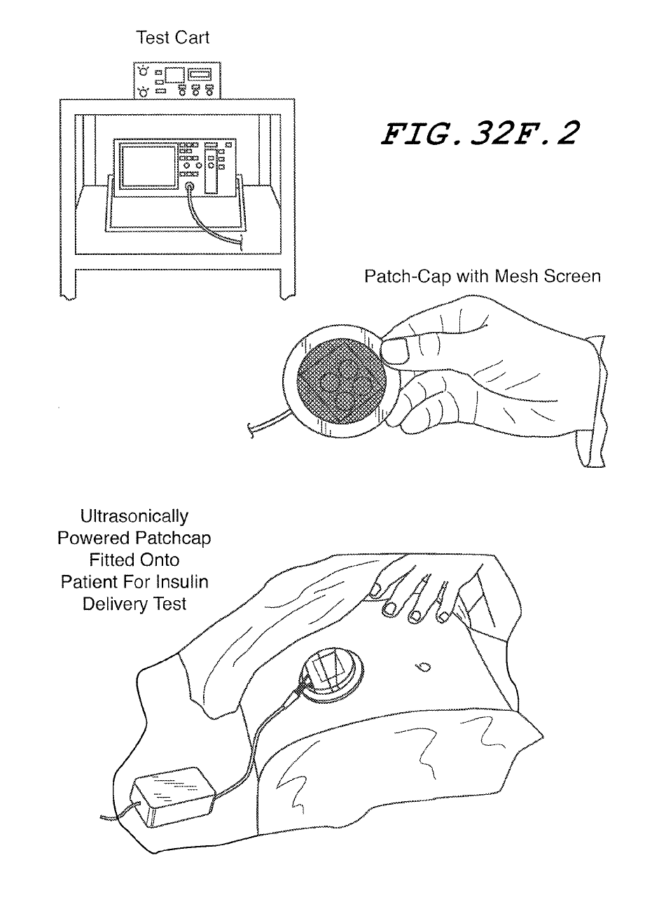

FIG. 32-G illustrates a Portable transdermal delivery device, coupled with a Patch-cap, powered by ultrasound for the delivery of insulin in the treatment of diabetes. A transdermal delivery device 76, either a flexible patch or a patch-cap containing insulin is affixed to the patient and held in place either through adhesives or through the use of a strap 84. The transdermal delivery device is connected electronically 204 to a control device 400 which may be fitted to the patients belt 250. The control device 400 transmits electrical energy to the transdermal delivery device 300, or more specifically to a transducer or array of transducers which are fitted onto or within the patch or patch-cap and cause insulin stored within the transdermal delivery device to become deposited onto the surface of the skin, through a transdermal delivery device fitted with a mesh screen or filter on its proximal end 50 or underside. The ultrasound causes the insulin to become deposited within the skin tissue and eventually lowers and regulates the glucose level of a diabetic patient, who may be either classified as type-1, type-2 or type-3 diabetes sufferer.

Referring now to FIG. 12, a flow chart for a method of the invention is illustrated. Embodiments of the method of the invention include, but are not limited to, utilization of the devices, dimensions, function, design and materials described in the device embodiments of the invention. In one embodiment, the method includes providing 90 a substance delivery device, providing 92 at least one separator at the proximal end of the delivery device, placing 94 the device adjacent to a living tissue or next to a material through which the substance my flow to access living tissue, and enabling 96 the release of the substance in the form of droplets to access the living tissue.

### Experiments

### Experiment 1

Increase in Speed of Absorption of Insulin when Propagated by Ultrasound, Using the Patch-Cap Construction Indicated in FIG. 8A and B, Using a Mesh Screen Vs. Without a Mesh Screen.

### Experiment Number: BKR-1000-124

Refer to FIG. 32F where a Patch-Cap active transdermal delivery device 300 is attached to a patient 250 and held in place with a strap. The Patch-Cap is loaded with 100 units of Lispro insulin (Humalog supplied by Eli Lilly Co.) and is powered by an ultrasonic applicator device 23 on a nearby table 255. The ultrasound is monitored by a computer 254 connected to an oscilloscope 252.

FIG. 32-F is the connection to a volunteer for Experiment-1, a test of transdermal delivery device, a Patch-cap, loaded with insulin and powered by ultrasound, with and without the use of a mesh screen.

FIG. 32-F.2 is an illustration of Experiment-1, a test of a transdermal delivery device. A Patch-Cap, loaded with insulin and powered by ultrasound, with the use of a mesh screen, as it was affixed to a test subject during the experiment. In this illustration a test Cart, consisting of an Oscilloscope mounted on the cart to an ultrasonic generator was placed in vicinity of a subject. A patch-Cap with a mesh screen on its bottom was affixed to the right side abdomen of the subject.

FIG. 32-F.3 is an illustration of the equipment used in Experiment-1.

FIG. 32-F.4 is an n illustration of a Patch-Cap loaded with 100 units of Lispro insulin, used in Experiment-1, a test of a transdermal delivery device, a Patch-cap, loaded with insulin and powered by ultrasound, with the use of a mesh screen. The Patch-Cap 300 is connected to a transducer array 301. At the bottom of the Patch-Cap 300 a mesh screen 302 is placed to form the insulin droplets onto the surface of the skin.

A male, Type-2 diabetic volunteer was used in both experiments, One day tested with the insulin loaded Patch-cap. The Same volunteer was test 4 days later with an insulin loaded Patch-cap fitted with a mesh screen. The 4-day wash out period was to allow the patient's glucose level to rebound back to it starting level, with not left over insulin from the first experiment. The goals of these experiments was to determine if there was a clinical benefit to a patch fitted with a mesh screen vs. one fitted without. An ultrasonically powered patch-Cap was chosen for these experiments.

TEST A: PATCH-CAP WITH NO MESH SCREEN

| Frequency: | 23 | kHz |
|---|---|---|
| Intensity: | 500 | mW/sq. cm |
| Ultrasonic Transmission | Alternating between 50 milliseconds | |
| | saw tooth and 50 milliseconds square | |
| | wave form. This alternation avoid | |
| | cavitation or over heating of the insulin | |
| | within the Patch-Cap. | |
| Placement upon Patient: | Right side of the abdomen | |
| Dimensions of Patch-Cap | 2.25 inch diameter absorbent pad area | |
| Absorbent pad used | Cellulose, 1 mil thickness | |
| Number of absorbent pads | One | |
| in Patch-Cap | | |
| Duration of Experiment: | 4 | hours |

| | |
|---|---|
| Filter Screen used | NONE |

The delivery pattern of the drug upon the surface of the skin corresponded to the pooling effect shown in FIG. 4B.

Results. No Mesh Screen.

The delivery rate was 7.2 units of insulin per hour. It took 4 hours to reduce the glucose of the patient form 165 mg/dl to 125 mg/dl, a drop of 40 points or just 10 mg/dl per hour.

TEST B: PATCH-CAP WITH MESH SCREEN

| Duration of Trial | 4 | hours |
|---|---|---|
| Delivery rate was | 7.2 units of insulin per hour | |
| Glucose reduction | −40 | mg/dl. |

The ultrasound intensity through the transducer coupler part for the Patch-cap was set according to the following settings:

| Frequency: | 23 | kHz |
|---|---|---|
| Intensity: | 500 | mW/sq. cm |
| Ultrasonic Transmission | Alternating between 50 milliseconds | |
| | saw tooth and 50 milliseconds square | |
| | wave form. This alternation avoid | |
| | cavitation or over heating of the | |
| | insulin within the Patch-Cap. | |
| Placement upon Patient: | Right side of the abdomen | |
| Dimensions of Patch-Cap | 2.25 inch diameter absorbent pad area | |
| Absorbent pad used | Cellulose, 1 mil thickness | |
| Number of absorbent pads | One | |
| in Patch-Cap | | |
| Duration of Experiment: | 4 | hours |

| | |
|---|---|
| Filter Screen used | a) Nylon |
| | b) 100 × 100 fibers per inch |
| | vertical & horizontal. |
| Source of Mesh Material | TSI Filtration Technologies |
| | 6148k Brookshire Blvd. |
| | Charlotte, NC. 28216 |

Results. With the Mesh Screen.

The delivery rate was 16.4 units of insulin per hour. In 35 minutes the glucose of the patient dropped from 165 mg/dl to 95 mg/di, a drop of 70 points in a little over half an hour. The trial had to be halted when the patient reached the range of a Health Normal, Non-Diabetic adult.

| Duration of Trial | 35 | minutes |
|---|---|---|
| Delivery rate was | 16.4 units of insulin per hour | |
| Glucose reduction | −70 | mg/dl. |

The glucose drop was highly significant with the mesh screen fitted Patch-Cap. In fact the patient illustrated an 8 point drop in glucose, in just the first 5 minutes when using the mesh fitted patch-Cap. The drug deposition upon the skin was as shown in FIG. 5B.

This experiment showed that a mesh screen placed at the bottom of a transdermal delivery device, either a flexible patch or apparatus such as the Patch-Cap, dramatically improves the absorption of the drug through the skin, while reducing the waste left upon the surface of the skin.

In fact, coupled with an ultrasonic propagation mechanism this experiment demonstrated significant advantages to the treatment of diabetes, through the use of a mesh screen affixed to the bottom of a transdermal delivery device or patch.

### Comparison and Observations

| | |
|---|---|
| Test-A with No Screen | Test-B with Screen |
| on Patch-Cap | on Patch-Cap |

| | | | | |
|---|---|---|---|---|
| Duration of Trial | 4 | hours | 35 | minutes |

| | | |
|---|---|---|
| Delivery rate was | 7.2 units of | 16.4 units of |
| | insulin per hour | insulin per hour |

| | | | | |
|---|---|---|---|---|
| Glucose reduction | −40 | mg/dl. | −70 | mg/dl. |

The Patch-Cap, powered by Ultrasound, using a mesh screen at the bottom was far more potent at glucose reduction than afforded by the same Transdermal delivery Device which did not employ the screen.

The Delivery Rate using the mesh was 2.27 times more efficient at releasing than insulin then the patch-cap without the mesh screen.

### Combination Ultrasonic Device and Transdermal Patch

The invention further includes a method for conducting the transport of active substances, including but not limited to pharmaceutical compositions, through the body surface of an individual. The method includes applying ultrasound through a transdermal delivery device which is attached with to a programmable ultrasonic regulator device, which itself is worn by the individual wherein said ultrasound is applied at a frequency and intensity and for a time period effective to enable movement of a therapeutic quantity of the active pharmaceutical composition from a transdermal delivery device, or transdermal patch, through the skin, for the purpose of effecting regulated, and timed drug delivery to the individual.

The method of can also include providing an ultrasound having a frequency in the range of about 20 kHz to 10 MHz, and having intensity in the range of about 0.01 W/cm.sup.2 to 5.0 W/cm.sup.2., and wherein the ultrasound is applied either in a continuous or pulsed manner.

The method can further include affixing or connecting the wearable, portable sonic device with a transdermal patch which provides the transdermal delivery of drugs or other substances to the individual. The connection can be effected via the use of a snap-on feature built into the transdermal patch, or by some other effective connector which provides a connection of the backbone of the patch with a transducer or array of transducers.

The method can further include providing that the wearable, portable sonic device is controllable through programmable settings for at least one of the following: the quantity of drug released by the device, the time interval of active ultrasonic drug delivery, the time interval between ultrasonic drug delivery, the frequency and intensity of the ultrasonic signal, the basal delivery schedule of drug dosing and the bolus delivery schedule of booster doses of a particular drug, with both automatic functions and a manual operation capability.

The invention further includes a delivery device for conducting the transport of active substances, including but not limited to pharmaceutical compositions, through the body surface of an individual, which is attachable with a programmable ultrasonic regulator device. The programmable ultrasonic regulator device is wearable by the individual wherein ultrasound is applied through the device at a frequency and intensity and for a time period effective to enable movement of a therapeutic quantity of the active pharmaceutical composition from a transdermal delivery device, or transdermal patch, through the skin or live tissue for the purpose of effecting regulated, and timed drug delivery to the individual. The delivery device can also contain a transducer assembly, holding a single or multiple transducers of any effective type including cymbal type, wherein the transducer assembly may be internal or external to the device.

The invention further includes an ultrasonic drug deliverer that uses a single transducer or an array of transducers, employed to deliver ultrasonic energy through a transdermal patch, wherein the array makes possible the application of the ultrasonic drug transport through a number of multiple skin transport sites. The drug deliverer avoids premature damage to the skin transport sites and effects the greatest quantity of deliverable drug from the patch, through the patients skin and into the bloodstream, in some embodiments, the multiple transducer elements in the drug deliverer transmit ultrasound at identical frequencies and intensity levels to each other. In some embodiments, the multiple transducer elements in the drug deliverer transmit ultrasound at differing frequencies and intensity levels to each other.

The invention includes an ultrasonic substance delivery transdermal patch, wherein the modified transdermal patch includes:
A) Patch Backbone and Sonic membrane:
   A backbone of the patch has a section including a membrane which will enable the effective transmission of the ultrasonic signal throughout the patch, said membrane possessing properties which will not interfere with the frequency or reduce the intensity of the ultrasonic transmission, wherein the membrane may be made of a material including saran, or such other polymeric compound which will similarly not interfere with the frequency and intensity of an ultrasonic transmission.
B) Absorbent Pad:
   An absorbent compound as a means for storing a substance, including but not limited to a medication, drug or nutrient compound within the patch, wherein an ultrasonic transmission though the patch acts to liberate the substance from the absorbent pad to be transported to the patient through skin permeation.
C) Semi-Permeable Film
   A semi-permeable film at the bottom of the patch, at the interface where the patch comes into contact with the patients skin. The semi-permeable film provides a means for delivering a stored substance including but not limited to a medication, drug or nutrient compound from within the patch to the patients skin surface only upon the active generation of ultrasonic transmissions through the patch thereby providing an On-Off function with the propagation of ultrasound through the patch, and a means of regulating the quantity of the substance or dose to the patient, i.e., the control of the delivered dose to the patient, wherein said semi-permeable film is composed of a material which provides osmotic by-pass, via ultrasonic propagation, or is composed of a membrane or film possessing perforations which expand in the presence of ultrasound and which contract when ultrasound is terminated, to enable substance delivery.
D) Gasket for providing a good seal to the skin
   A gasket around the backbone of the patch, as means of preventing air from reaching under the patch and interfering with the intensity of the ultrasonic transmission through the patient's skin and for preventing leakage of the drug contained within the patch.

In some embodiments of the transdermal patch, the semi-permeable film may be composed of materials including but not limited to the following materials:
Membranes:
   CTA (Cellulose Tri-Acetate)
   TFC (Thin Film Composite) sometimes labeled as TFM (Thin Film Membrane).
Reverse Osmosis membranes made from semi permeable material such as:
   Cellulose tri Acetate
   Composite polyamide
Membrane films using;
   Pierced membranes
   Spiral wound membranes
Commercial examples of semi-permeable films include:
   Hytrel@
   Surlyn@
   Crastin@
   Imron@
   CA (cellulose acetate)

The at least one absorbent pad in the transdermal patch may include materials including, but is not limited to, the following list of materials:

| | |
|---|---|
| Cellulose Fiber Pad | Cotton |
| Natural Sponge | Woven Cloth Fabrics |
| Polyurethane foams | Polyisocynurate Foams |
| Non-Woven Cloths | Fumed Silica |
| Starch | Corn Meal |
| Wood Pulp fibers | Collagen Pads |
| Poly methyl methacrylate | Polyvinyl alcohol |
| Poly vinyl pyrralidine | Poly acrylic acid |
| Poly (2-hydroxy ethyl methacrylate | Polyacrylamide |
| Poly ethylene glycol | Polylactides(PLA) |
| Polyglycolides(PGA) | Nylon |
| Poly(lactide-Co-glycolides) | Polypropolene |
| Polycarbonate | Chitosan |

Poly (N-isopropylacrylamide)
Co-Polymer formulations of Poly methacrylic acid and Poly ethylene
glycol
Co-Polymer formulations of Poly acrylic acid and Poly (N-isopropyl-
acrylamide)
Hyrdogels, e.g. Polyacrylamide, poly(propylene oxide
Pluronic polyols family of gel materials, e.g. Pluronic-chitosan hydrogels
Silica gels

It is to be understood that the at least on pad could also be made of any other natural or synthetic material, which will act to absorb the drug compound and be able to release the drug upon ultrasonic excitation.

In some embodiments, the use of an absorbent pad is made to provide extended delivery of the substance via the manipulation of the thickness of the absorbent material, or through the selection of materials with increased absorbency power, thereby enabling the absorbent pad to hold and reserve greater quantities or doses of the substance to be delivered, for a longer period of time.

In some embodiments of the invention, the delivery rate of a substance from the transdermal patch can be adjusted due to the use of an absorbent pad via the manipulation of the thickness of the absorbent material, or through the selection of materials with increased or decreased absorbency power, thereby enabling the absorbent pad to liberate the substance at differing delivery rates form the patch.

In some embodiments of the transdermal patch the use of an absorbent pad provides enhanced resistance to incidental contact between the stored substance and other materials or compounds within the patch construction which could contaminate or degrade the substance, including adhesives used in the fabrication of the patch or to adhere the patch to the patients skin surface.

In some embodiments, the invention further includes, a means of providing regulated and controlled doses of insulin and other medications for the treatment of diabetes, involving a wearable ultrasonic transmitter which is connected to a transdermal patch wherein the patch has been loaded with insulin or other medication for the treatment of diabetes. The combination device acts to regulate the dose delivered to a diabetic patient for the purpose of reducing and controlling serum glucose levels in the diabetic patient.

In some embodiments, the invention includes a combination system that includes a wearable ultrasonic transmitter which is connected to a transdermal patch for the purpose of providing regulated and controlled doses of insulin and other medications for the treatment of diabetes, wherein the insulin loaded patch is used either in conjunction with or in replacement of oral diabetic medication, for night time use, daytime use or both, for the purpose of reducing and controlling serum glucose levels in a diabetic patient.

The invention further includes an enhanced ultrasonic drug delivery transdermal patch suitable for ultrasonic drug delivery, containing an absorbent compound as a means for storing a substance, including but not limited to medication, drugs or nutrient compounds within the patch, wherein the absorbent compound is made to be more resonance compatible with the frequency and intensity of the ultrasonic transmission by pre-treating the absorbent compound to improve its sonic attenuation properties by reducing the quantity of air or gas trapped within the absorbent by: Freezing the absorbent material, and Vacuum drying the absorbent material and/or by Pre-treating the material with sonic energy to remove any impurities within the absorbent material, prior to the application of the substance to the material.

The invention further includes embodiments of a means of instilling a sonic memory into materials used as the semi-permeable film layer of a transdermal patch, wherein the materials are subjected to ultrasound at the desired reactant frequency and intensity levels, while being formulated and cast into a film or membrane state, for a period of time as to make that film or membrane activate its reverse osmosis properties or pore dilation in response to a ultrasonic signal of the same amplitude, frequency and intensity level used during the formulation process.

The invention further includes a modified transdermal delivery device which incorporates a mesh screen at the bottom of the transdermal delivery device, which contacts to the skin, for the purpose of avoiding drug pooling, improving drug absorption, and the speed of absorption of the drug.

The invention further includes a flexible transdermal patch delivery device which incorporates a mesh screen at the bottom of the transdermal delivery device, which contacts the skin, for the purpose of avoiding drug pooling, improving drug absorption, and increasing the speed of absorption of the drug.

The invention further includes embodiments of a transdermal delivery cap or patch-cap delivery device which incorporates a mesh screen at the bottom of the device, which contacts the skin, for the purpose of avoiding drug pooling, improving drug absorption, and increasing the speed of absorption of the drug.

The invention further includes a transdermal delivery device which incorporates a mesh screen attachment in the form of a cap which can be added to the underside or proximal end of a transdermal delivery device for the purpose of avoiding drug pooling, improving drug absorption, and increasing the speed of absorption of the drug.

Having described the invention in the above detail, those skilled in the art will recognize that there are a number of variations to the design and functionality for the device, but such variations of the design and functionality are intended to fall within the present disclosure.

I claim:

1. A method for conducting the transport of active substances, including but not limited to pharmaceutical compositions, through the body surface of an individual, comprising applying ultrasound through a transdermal delivery device which is affixed to a programmable ultrasonic regulator device, which itself is worn by the individual wherein said ultrasound is applied at a frequency and intensity and for a time period effective to enable movement of a therapeutic quantity of said active pharmaceutical composition from a transdermal delivery device, or transdermal patch, through the skin, for the purpose of effecting regulated, and timed drug delivery to the individual.

2. The method of claim 1, wherein said ultrasound has a frequency in the range of about 20 kHz to 10 MHz. and intensity of said ultrasound is in the range of about 0.01 W/cm.sup.2 to 5.0 W/cm.sup.2., wherein the ultrasound is applied either in a continuous pulsed manner.

3. The method of claim 1, wherein the wearable, portable sonic device is affixed onto or connects to a transdermal patch which provides the transdermal delivery of drugs or other substances to the individual, said connection being effected via the use of a snap-on feature built into the transdermal patch, or by some other effective connector means which provides the backbone of the patch connects to attach itself to a transducer or array of transducers.

4. The method of claim 1, wherein the wearable, portable sonic device is controllable through programmable settings such as to the quantity of drug released by the device, the time interval of active ultrasonic drug delivery, the time interval between ultrasonic drug delivery, the frequency and intensity of the ultrasonic signal, the basal delivery schedule of drug dosing and the bolus delivery schedule of booster doses of a particular drug, with both automatic functions and a manual operation capability.

5. Apparatus as in claim 1, which contains a transducer assembly, holding a single or multiple transducers of any effective type including cymbal type, wherein the transducer assembly may be internal or external to the device.

6. A means of conveying ultrasonic drug delivery through the use of a single transducer or an array of transducers, employed to deliver ultrasonic energy through a transdermal patch, wherein the array makes possible the application of the ultrasonic drug transport through a number of multiple skin transport sites, for the purpose of avoiding premature damage to the skin transport sites and effecting the greatest quantity of deliverable drug from the patch, through the patients skin and into the bloodstream.

7. A means of conveying ultrasonic drug delivery as claimed in claim 6, wherein the multiple transducer elements transmit ultrasound at identical frequencies and intensity levels to each other or alternatively transmit ultrasound at differing frequencies and intensity levels to each other.

8. A means of enhanced ultrasonic substance delivery employing a modified transdermal patch, wherein the modified transdermal patch comprises:
A) Patch Backbone and Sonic membrane:
A backbone of the patch has a section composed of a membrane which will enable the effective transmission of the ultrasonic signal throughout the patch, said membrane possessing properties which will not interfere with the frequency or reduce the intensity of the ultrasonic transmission, wherein said membrane may be composed of saran, or such other polymeric compound which will similarly not interfere with the frequency and intensity of an ultrasonic transmission.
B) Absorbent Pad:
An absorbent compound as a means for storing a substance, including but not limited to a medication, drug or nutrient compound within the patch, wherein an ultrasonic transmission through the patch acts to liberate the substance from the absorbent pad to be transported to the patient through skin permeation.
C) Semi-Permeable Film
A semi-permeable film at bottom of the patch, at the interface where the patch comes into contact with the patients skin, said semi-permeable film providing a means for delivering a stored substance, including but not limited to a medication, drug or nutrient compound from within the patch to the patients skin surface only upon the active generation of ultrasonic transmissions through the patch thereby providing an On-Off function with the propagation of ultrasound through the patch, and a means of regulating the quantity of the substance or dose to the patient, i.e., the control of the delivered dose to the patient, wherein said semi-permeable film is composed of a material which provides osmotic by-pass, via ultrasonic propagation, or is composed of a membrane or film possessing perforations which expand in the presence of ultrasound and which contract when ultrasound is terminated, to enable substance delivery.
D) Gasket for providing a good seal to the skin
A gasket around the backbone of the patch, as means of preventing air from reaching under the patch and interfering with the intensity of the ultrasonic transmission through the patient's skin and for preventing leakage of the drug contained within the patch.

9. A transdermal Patch according to claim 8, wherein said semi-permeable film may be composed of, but not limited to the following materials:
Membranes:
CTA (Cellulose Tri-Acetate)
TFC (Thin Film Composite) sometimes labeled as TFM (Thin Film Membrane).
Reverse Osmosis membranes made from semi permeable material such as:
Cellulose tri Acetate
Composite polyamide
Membrane films using;
Pierced membranes
Spiral wound membranes
Commercial examples of semi-permeable films include:
Hytrel®
Surlyn®
Crastin®
Imron®
CA (cellulose acetate)

10. An absorbent pad according to claim 8 which may include, but is not limited to the following list of materials:

| | |
|---|---|
| Cellulose Fiber Pad | Cotton |
| Natural Sponge | Woven Cloth Fabrics |
| Polyurethane foams | Polyisocynurate Foams |
| Non-Woven Cloths | Fumed Silica |
| Starch | Corn Meal |
| Wood Pulp fibers | Collagen Pads |
| Poly methyl methacrylate | polyvinyl alcohol |
| Poly vinyl pyrrolidine | Poly acrylic acid |
| Poly (2-hydroxy ethyl methacrylate | Polyacrylamide |
| Poly ethylene glycol | Polylactides (PLA) |
| Polyglycolides (PGA) | Poly (lactide-Co-glycolides) |
| Polycarbonate | Chitosan |

Poly (N-isopropylacrylamide)
Co-Polymer formulations of Poly methacrylic acid and Poly ethylene
glycol
Co-Polymer formulations of Poly acrylic acid and Poly (N-isopropyl-
acrylamide)
Hyrdogels, e.g. Polyacrylamide, poly (propylene oxide
Pluronic polyols family of gel materials, e.g. Pluronic-chitosan hydrogels
Silica gels
Or any other natural or synthetic material, which will act to absorb the drug, compound and be able to release the drug upon ultrasonic excitation.

11. A means of enhanced ultrasonic drug delivery employing a modified transdermal patch suitable for ultrasonic drug delivery, containing an absorbent compound as a means for storing a substance, including but not limited to medication, drugs or nutrient compounds within the patch, wherein the absorbent compound is made to be more resonance compatible with the frequency and intensity of the ultrasonic transmission by pre-treating the absorbent compound to improve its sonic attenuation properties by reducing the quantity of air or gas trapped within the absorbent by:
a) Freezing the absorbent material, and Vacuum drying the absorbent material or by:
b) Pre-treating the material with sonic energy to remove any impurities within the absorbent material, prior to the application of the substance to the material.

12. A transdermal Patch according to claim 8, wherein said use of an absorbent pad is made to provide extended delivery of the substance via the manipulation of the thickness of the absorbent material, or through the selection of materials with increased absorbency power, thereby enabling the absorbent pad to hold and reserve greater quantities or doses of the substance to be delivered, for a longer period of time.

13. A transdermal Patch according to claim 8, wherein the delivery rate of a substance from said transdermal patch can be adjusted due to the said use of an absorbent pad via the manipulation of the thickness of the absorbent material, or through the selection of materials with increased or decreased absorbency power, thereby enabling the absorbent pad to liberate the substance at differing delivery rates form the patch.

14. A means of instilling a sonic memory into materials used as the semi-permeable film layer of a transdermal patch wherein the materials are subjected to ultrasound at the desired reactant frequency and intensity levels, while being formulated and cast into a film or membrane state, for a period of time as to make that film or membrane activate its reverse osmosis properties or pore dilation in response to a ultrasonic signal of the same amplitude, frequency and intensity level used during the formulation process.

15. A transdermal Patch according to claim 8, wherein said use of an absorbent pad provides enhanced resistance to incidental contact between the stored substance and other materials or compounds within the patch construction which could contaminate or degrade the substance, including adhesives used in the fabrication of the patch or to adhere the patch to the patients skin surface.

16. A means of providing regulated and controlled doses of insulin and other medications for the treatment of diabetes, involving a wearable ultrasonic transmitter which is connected to a transdermal patch as claimed in claim 8, wherein the patch has been loaded with insulin or other medication for the treatment of diabetes and said combination device acts to regulate the dose delivered to a diabetic patient for the purpose of reducing and controlling serum glucose levels in said diabetic patient.

17. A combination system as claimed in claim 16, comprising a wearable ultrasonic transmitter which is connected to a transdermal patch as claimed in claim 8, for the purpose of providing regulated and controlled doses of insulin and other medications for the treatment of diabetes, wherein the insulin loaded patch is used either in conjunction with or in replacement of oral diabetic medication, for night time use, daytime use or both, for the purpose of reducing and controlling serum glucose levels in said diabetic patient.

18. A modified transdermal delivery device which incorporates a mesh screen at the bottom of the transdermal delivery device, which contacts to the skin, for the purpose of avoiding drug pooling, improving drug absorption, and the speed of absorption of the drug.

19. A modified transdermal delivery device which incorporates a mesh screen at the bottom of the transdermal delivery device, which contacts the skin, for the purpose of avoiding drug pooling, improving drug absorption, and increasing the seed of absorption of the drug, wherein the transdermal delivery device is a flexible transdermal patch.

20. A modified transdermal delivery device which incorporates a mesh screen at the bottom of the device, which contacts the skin, for the purpose of avoiding drug pooling, improving drug absorption, and increasing the speed of absorption of the drug, wherein the transdermal delivery device is a transdermal delivery cap or patch-cap device.

21. A modified transdermal delivery device which incorporates a mesh screen attachment in the form of a cap which can be added to the underside of a transdermal delivery device for the purpose of avoiding drug pooling, improving drug absorption, and increasing the speed of absorption of the drug.
